(19) United States
(12) Patent Application Publication
(10) Pub. No.: US 2019/0274547 A1
(43) Pub. Date: Sep. 12, 2019

(54) SYSTEM AND METHOD OF NONCONTACT VITAL SIGNS MONITORING THROUGH LIGHT SENSING

(71) Applicant: The Board of Regents for Oklahoma State University, Stillwater, OK (US)

(72) Inventors: SABIT EKIN, STILLWATER, OK (US); HISHAM FADLALLAH M. ABUELLA, STILLWATER, OK (US)

(73) Assignee: The Board of Regents for Oklahoma State University, Stillwater, OK (US)

(21) Appl. No.: 16/295,603

(22) Filed: Mar. 7, 2019

Related U.S. Application Data
(60) Provisional application No. 62/639,524, filed on Mar. 7, 2018.

(51) Int. Cl. A61B 5/00; A61B 5/08; A61B 5/113; A61B 5/04; A61B 5/0205

(52) U.S. Cl. CPC A61B 5/0077; A61B 5/0816; A61B 5/7225; A61B 5/7207; A61B 5/113; A61B 5/02416; A61B 5/0205; A61B 5/0064; A61B 5/7257; A61B 5/7278; A61B 5/7221; A61B 5/04014

(57) ABSTRACT

According to an embodiment there is taught herein a system and method of noncontact human vital signs monitoring using light sensing (also referred to generally herein as visible light sensing (VLS)) that can, in some embodiments, measure in real-time breathing (respiration) and heartbeat rates without body contact. One variation comprises a photo-detector (PD) sensor, which measures signal power level of the reflected light signal from the patient's body and an embedded processing system for the signal processing and filtering on the received light signal. The technology taught herein has the potential to address numerous conditions where the use of contact-based devices may be problematic.

### CROSS REFERENCE TO RELATED APPLICATIONS

This application claims the benefit of U.S. Provisional Patent Application Ser. No. 62/639,524 filed on Mar. 7, 2018, and incorporates said provisional application by reference into this document as if fully set out at this point.

### TECHNICAL FIELD

This disclosure relates generally to systems and methods of monitoring of patient vitals and, more particularly, to systems and methods of noncontact vitals monitoring using light sensing, e.g., visible light, infrared, ultraviolet, etc.

### BACKGROUND

Monitoring human body vitals is critical for accurate diagnosis of diseases and for deciding appropriate treatment and/or therapy methods. In particular, respiration rate and heart rate are of importance for heart and lungs related diseases. Anomalies in vitals can indicate major illness. Many vitals monitoring systems have been introduced to get the most accurate results either contact- or noncontact-based. In addition, most of the state-of-the-art systems for the vital signs measurements are based on contact sensors such as probes, chest straps, pulse oximeters and blood pressure cuffs. However, contact-based sensors may not be user friendly at times and, as a consequence, many researchers are looking into alternative noncontact approaches. In that regard, researchers have been pursuing forms of non-contact monitoring of vital signs since the 1970's. Several important medical scenarios motivate a continued and determined research effort in this area; (1) cases where patients have delicate or injured skin such as low-birthweight, pre-termn newborns, patients in burn units, (2) cases where wiring and/or ECG leads endanger or perturb the patient such as infants that might potentially be subject to sudden-infant-death-syndrome (SIDS) and sleep apnea, (3) other situations which might involve financial concerns, e.g, hospital-acquired infections (HAIs) due to cross-contamination among patients (due, e.g. to reusable ECG leads).

Prior art methods of using a camera/image processing techniques to estimate vitals are known and include, as one example, using a 3D machine vision method and infrared light (dynamic photometric stereo). In another approach, the authors used thermal imaging to measure human cardiac pulse at a distance without any contact device. Others have used normal cameras based on automatic face tracking to measure the change of skin color for cardiac pulse measurements, also referet to as imaging photoplethysmography (iPPG). Camera-based (image processing) real-time techniques have processing time and power issues. Moreover, the security and privacy concerns remain problematic.

Another prior art approach involved the use of RF signals to measure the change of the phase of the reflected signal. For example, one approach involved a device that can measure breathing and heart rates using a radar technique called FMCW (Frequency Modulated Carrier Waves). Others have proposed an existing WiFi network for tracking vital signs of breathing and heart rates during sleep. Still others have presented an algorithm called RELAX to improve the performance of noncontact RF vital sign detection based on Doppler RADAR.

However, prior art approaches are not without their problems. For example, using RF signals has some issues in channel modeling because of the multipath and the reflected paths from different objects. In addition, because of health concerns a low power transmitter is used that can affect the accuracy of the estimated result. Finally, using RF signals will consume a bandwidth and will cause RF interference to other devices using the same band in the same environment. RF based methods may lead to electromagnetic interference (EMI) in critical electrical circuits (e.g., life-saving medical equipment). They can also present safety concerns in terms of long-term exposure and limited range, and therefore aren't universally accepted in hospitals.

Additionally, conventional approaches that require direct contact with the patient's body might use devices that irritate the patient's skin. Also, patients might feel uncomfortable (e.g., anxious, nervous, and/or excited) when sensors are placed into contact with their body. Such feelings can affect the patient's breathing and heartbeat measurements and mislead the patient and/or healthcare provider who might rely on such measurements.

As such, what is needed is a system and method of measuring certain patient parameters without contacting the patient that does not suffer from the disadvantages of the prior art.

Before proceeding to a description of the present invention, however, it should be noted and remembered that the description of the invention which follows, together with the accompanying drawings, should not be construed as limiting the invention to the examples (or embodiments) shown and described. This is so because those skilled in the art to which the invention pertains will be able to devise other forms of this invention within the ambit of the appended claims.

### SUMMARY

Taught herein are various embodiments of a system for noncontact monitoring of a subject's vitals that comprises of a photo-detector (PD) sensor, a processing/display unit and a light source (either ambient or special light that operated in different part of the spectrum). One of the main advantages of this approach is that it uses visible light, which is safer than RF based noncontact health monitoring system. One proposed system is capable of measuring and estimating the heart and breathing rates of human body using the variation of the received light intensity from the reflection of light source on the human body. In one embodiment, the use of LED received power level for vital monitoring has not been studied heretofore.

According to a first embodiment, there is provided a novel system and method monitoring a subject's vitals method using visible light sensing (VLS). Measurements of breathing (respiration) and heartbeat rates can be acquired without device that directly contacts a patient's body. Conventional techniques and hardware for tracking body vitals require body contact and most of these techniques are intrusive. Note that for purposes of the instant disclosure, VLS will be broadly interpreted to refer to both light within the traditional visual spectrum as well as light that might be outside of the usual range, e.g., infrared and ultraviolet.

One embodiment consists of a photo-detector (PD) sensor, which measures the signal power level of the light signal reflected from the patient's body and an embedded processing system for signal processing and filtering the received light signal. Changes in the heartbeat and breathing can be monitored in a real-time mode after filtering the signal received from the PD. This method's accuracy has been shown to be comparable (with 95% accuracy according to one embodiment) to the state of the art vitals monitoring systems such as Pulse Oximeter. The instant reflected light approach, unlike RF based noncontact monitoring methods (RADAR sensors), does not penetrate the human body as RF signals can and, hence, does not cause the same sorts of safety concerns for long-term usage. In addition, various embodiments are safe and power-efficient, which can be used to monitor patient vitals either at home or at medical centers and are safe for use on newborns.

According to another embodiment, there is provided a system and method of determining patient vitals generally as described above, but wherein the light is outside the visible light spectrum, e.g., infrared or ultraviolet.

The foregoing has outlined in broad terms some of the more important features of the invention disclosed herein so that the detailed description that follows may be more clearly understood, and so that the contribution of the instant inventors to the art may be better appreciated. The instant invention is not to be limited in its application to the details of the construction and to the arrangements of the components set forth in the following description or illustrated in the drawings. Rather, the invention is capable of other embodiments and of being practiced and carried out in various other ways not specifically enumerated herein. Finally, it should be understood that the phraseology and terminology employed herein are for the purpose of description and should not be regarded as limiting, unless the specification specifically so limits the invention.

### BRIEF DESCRIPTION OF THE DRAWINGS

These and further aspects of the invention are described in detail in the following examples and accompanying drawings.

FIG. 1 illustrates a schematic drawing of an embodiment.

FIG. 2 contains an operating logic suitable for use with an embodiment.

FIG. 3 contains a schematic drawing that illustrates raw data of the sort that might be collected by an embodiment.

FIG. 4 contains typical filter frequency responses for two different kinds of patient vitals.

FIG. 5 contains exemplary data after filtering.

FIG. 6 contains a plan-view schematic of an embodiment.

FIG. 7 contains an example of a frequency domain view of measured light amplitude values after filtering to emphasize breathing and heartrate.

FIG. 8 provides an example illustration of heart and breathing error percentage as a function of distance from the patient.

FIG. 9 contains a schematic illustration of the received reflected signal power to the interference and noise ratio (SINR) at different positions from the PD and light source.

FIG. 10 illustrates heart and breathing error percentage and estimation variance for different data window sizes according to an embodiment.

FIG. 11 contains an illustration of heart and breathing error percentage for a Fast Fourier Transform (FFT) window length of 2048 samples (data window size) in different scenarios for a single subject.

### DETAILED DESCRIPTION

While this invention is susceptible of embodiment in many different forms, there is shown in the drawings, and will herein be described hereinafter in detail, some specific embodiments of the instant invention. It should be understood, however, that the present disclosure is to be considered an exemplification of the principles of the invention and is not intended to limit the invention to the specific embodiments or algorithms so described.

### System Setup

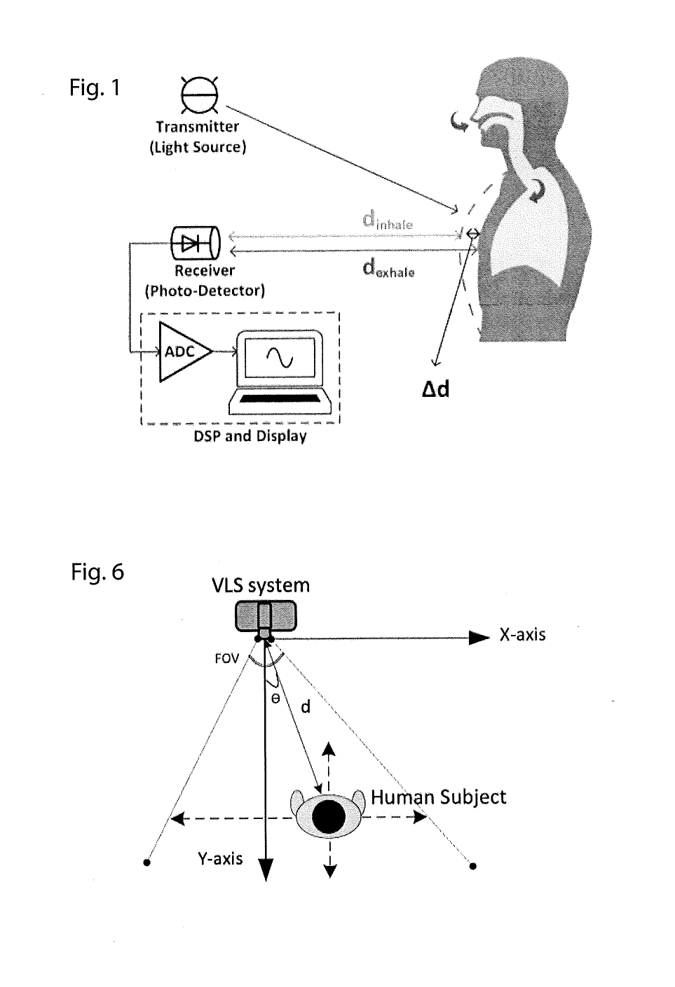

An embodiment is depicted in FIGS. 1 and 6. By way of explanation, the two positions noted in this figure indicate instances where the patient has inhaled or exhaled. The Δd differential shown in the figure indicates that the received power at the PD will be different for the two positions. Therefore, for the case when the human is exhaling air, the distance covered by the light wave is

d_{LED}+d_{PD}+2Δd,

while in the case of inhaling, the distance is decreased by approximately the same amount, 2Δd. In addition, d_{LED} represents the fixed distance between the subject and the light source and d_{PD} represents the relatively fixed distance between the subject and the PD, both d_{LED} and d_{PD} assume that the subject is in his or her inhaling position.

The transmitter/light source could be, by way of example only, ambient lighting (e.g., sunlight from a window, indoor lighting fixtures including fluorescent and tungsten lights, etc.), a specialized source (e.g., a LED array, a focused lamp, etc.), or some combination of the foregoing.

Note that, in some embodiments, a reflective patch, sticker or other device might be affixed to the patient's chest or upper torso area to increase the power of the reflected signal. This might be especially useful if the ambient light level is low. In some embodiments the sticker could be any light-reflective material, retro-reflective material, etc. In some cases, it might be useful to specify that the subject wear a specific color shirt or blouse to improve reflectivity (e.g., white, light grey, light blue, etc.).

The computing and display unit (the digital signal processor/DSP of FIG. 1) could be, by example only, a microcontroller, a computer (laptop or desktop) with attached monitor, a mobile smart phone, etc. The computational requirements of some embodiments are not particularly demanding so a smart phone or other handheld computing device could serve as the computational DSP engine. In one embodiment, the device used to convert the analog signal from the PD to a digital signal for the RaspberryPi™.

The ADC/analog to digital converter might be incorporated within the photo-detector or the computing engine/DSP. In some embodiments, Python script can be used to implement the signal processing algorithms within the DSP.

The photo-detector or photo sensor might be either wired to the or wirelessly in electronic communication with the DSP. Typically a digital camera is not used but, instead, a photo-detector is used, but the choice of such a sensor to suit a particular environment is well the capabilities of one of ordinary skill in the arts. The photo-detector might be equipped with optics/lenses to allow it to be focused on a limited region of the subject's body. Such arrangements are well known to those of ordinary skill in the art and need not be described here.

According to one embodiment, a Thorlabs™ PDA100A photo-detector, a RaspberryPi miniature computer, an ADC (Pi-Plate™) circuit, an off-the-shelf fixed power LED light source, and display unit were used to construct a working system, although other hardware configurations are certainly possible. The digital light reflectance data in this example stored within the RaspberryPi for subsequent analysis and processing by a separate computer.

In some embodiments, the following conditions are recommended in order to improve the quality of the measured vital signs:
The patient's body is to remain at least approximately stationary and in the same position/orientation during measurement.
The light source power is constant and does not flicker or otherwise vary in amplitude within the main frequency range of interest (e.g., 0.08 Hz-3 Hz).
The system should begin giving reliable estimations after 30 seconds of measurements.
The subject patients are asked to wear a grey color or other light colored shirt to reflect the light.
The system is operated in a controlled setting, i.e., excessive body movements and talking are avoided.

### Theory of Operation

A critical aspect in this embodiment is extracting the breathing and heart beat frequency components from a noisy time domain light signal. This is done in an embodiment by using signal processing algorithms applied to the received signals. Similar signal processing algorithms of the sort used herein have been used other contexts, e.g., in previous RF-based vitals monitoring studies.

One of the most used and adopted channel models for the line-of-sight (LOS) light transmission is the Lambertian model. The Lambertian model contains variables that describe the variation in the received light signal as a function of the transmitter power, distance between light source and PD, optical PD size, PD field of view (FOV) (which is the region of space where the detector can detect any light entering it) and incidence angle.

In order to simplify the calculations, it will be assumed that all the parameters except the distance between the PD and subject will remain constant during any vitals measurement test. A simplified model for the received signal power-distance relation as follows:

Pᵣ(t)=K[d(t)]^{-γ},

where Pᵣ(t) is the received signal power in watts is a function of time t, d(t) is the distance between the transmitter and the receiver in meters. The path-loss exponent depends on the environment conditions, such as reflectiveness of materials, humidity, etc. Typically, the range of path-loss exponent lies between 1 and 6. Notice that for a constant value of K and, the received power changes with the distance. The greater the distance between the transmitter (light source, e.g. LED) and receiver (PD), the lower the received power will be. Most importantly, variations in the distance (d) with certain periodicity result in similar variations in the received power with the same periodicity. In other words, the previous equation shows that one can identify variations in d due to breathing (inhaling and exhaling) and heartbeats, by measuring the resulting variations in the amplitude (power) of the received (reflected) signal. Such relation between the received signal power and distance serves as the foundation of the instant embodiment. Consequently, the periodicity of variations in the received power are due to small physical movements of human body (chest). These small movements are used to extract the rates for breathing and heartbeats.

As shown in FIG. 6, the position of the subject will affect the received signal power Pᵣ, since the distance d and the incidence angle θ between the subject and the PD will change. As known from Lambertian model the incidence angle θ will vary the received power with a cos(θ) factor where maximum power is received at θ=0°.

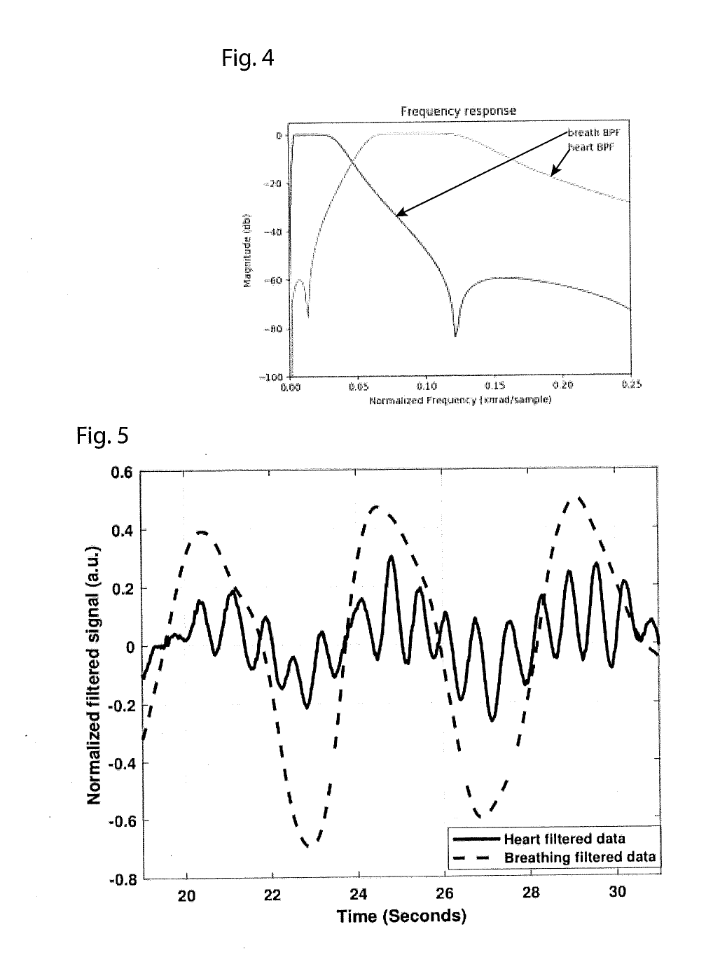

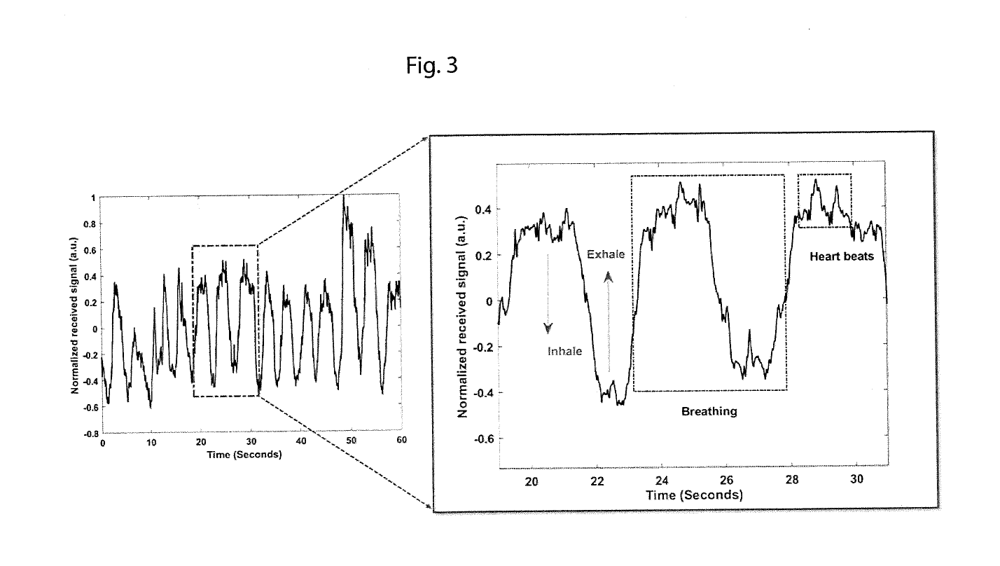

In this embodiment, an unmodulated light signal (generally ambient light but other options are certainly possible) is transmitted toward the subject, where it is amplitude modulated by the periodic physiological movements of the subject and reflected back to the receiver (i.e., the PD). The VLS receiver captures the reflected light intensity signal and processes it to extract the appropriate signal components. As shown in the example of FIG. 3, the peaks correspond to the times when the subject has inhaled, whereas the valleys correspond to the exhale position which, as might be expected, reflects the greater distance the signal has traveled and the correspondingly lower signal power. In addition to the breathing peaks and troughs, the breathing signal can be observed to be mixed with the heart beats and small heart beats fluctuations can also be easily noticed.

### Signal Processing Algorithms

In this section, the operation and flow of the breathing and heart rates estimation algorithms are provided. The problem here is that we need a good digital signal processing (DSP) algorithm to detect the small heartbeats from the data received to monitoring human vitals.

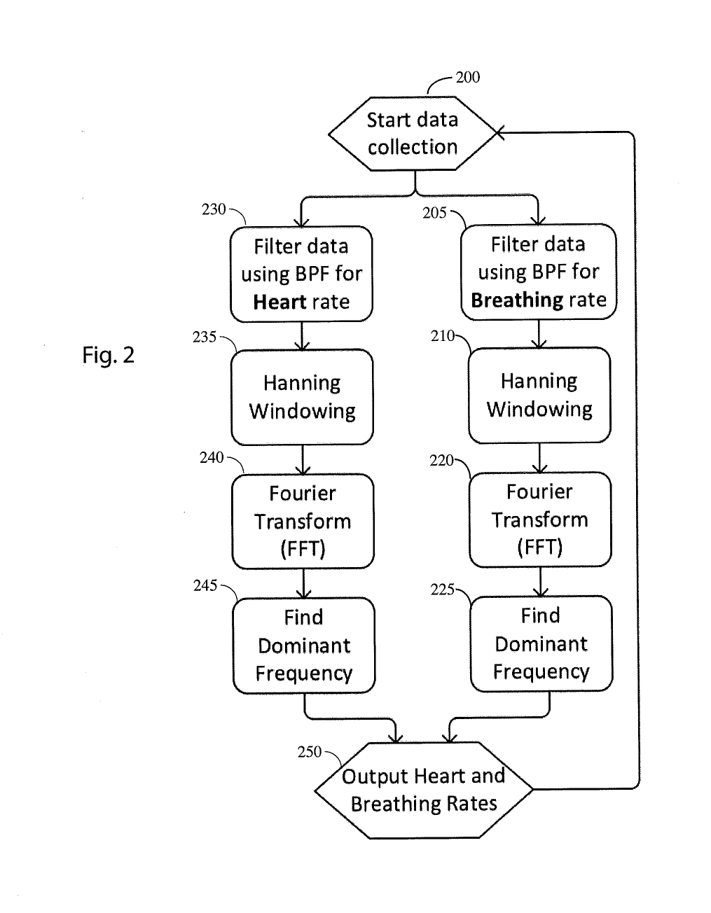

FIG. 2 contains an embodiment of a flow diagram of an algorithm suitable for use with the teachings presented herein. This example operating logic contains branches for estimating both heart and breathing rates.

Because breathing and heartbeats tend to be predominantly periodic events, the frequencies associated with them can often be identified and extracted by performing a Fourier analysis of the recorded amplitude data. Often a fast fourier transform (FFT) will be used to analyze the sample data but any methodology that is designed to identify periodic events might be used (e.g., a Walsh transform, general time series analysis, etc.). A FFT is especially useful because it can be quickly calculated to yield real time estimates of the parameters of interest. In addition, because breathing and heart beats occupy different frequencies, frequency domain operations may allow both to be recovered from noisy data via separate filtering operations. In the flow diagram of FIG. 2 (discussed below), separate branches are shown for breathing (boxes 205-225) and heart rate (boxes 230-245). The breathing (or heart) rate component is estimated in this embodiment by detecting the largest frequency component in the band of normal breathing (or heart) rate for an adult human (this can be tailored to other age ranges).

### Heart and Breathing Rates Estimation Algorithm

One approach is as follows. First, the raw amplitude data (box 200) is filtered with an infinite impulse response (IIR) band-pass filter (BPF) of appropriate passband range for adult breathing rate (box 205) or heart rate (box 230). In some embodiments an infinite impulse response (IIR) Chebyshev Type 2 band-pass filter (BPF) of appropriate passband could be used with the passband potentially being customized for the particular subject. The time domain light intensity signal in some cases might be band-pass filtered to limit the data to around 5 to 40 breaths per minute and 50-160 beats per minute (BPM) for breathing and heart rates, respectively. Obviously, the range of frequencies permitted by the band-pass filter(s) could be adjusted to accommodate different individuals or species, as the case may be.

The effect of band-pass filtering the data in the current example is shown in FIG. 5. After filtering the data, each dataset was multiplied with a Harming window (boxes 210 and 235) before transforming to the frequency domain (boxes 220 and 240) to remove the spectral leakage from the endpoints of the FFT process (i.e., reduce the edge effects associated with the start and end of the data). Of course, Hanning windows are just one approach to applying end tapers to reduce artifacts in the frequency domain and those of ordinary skill in the art will be readily to select an acceptable approach in a given situation.

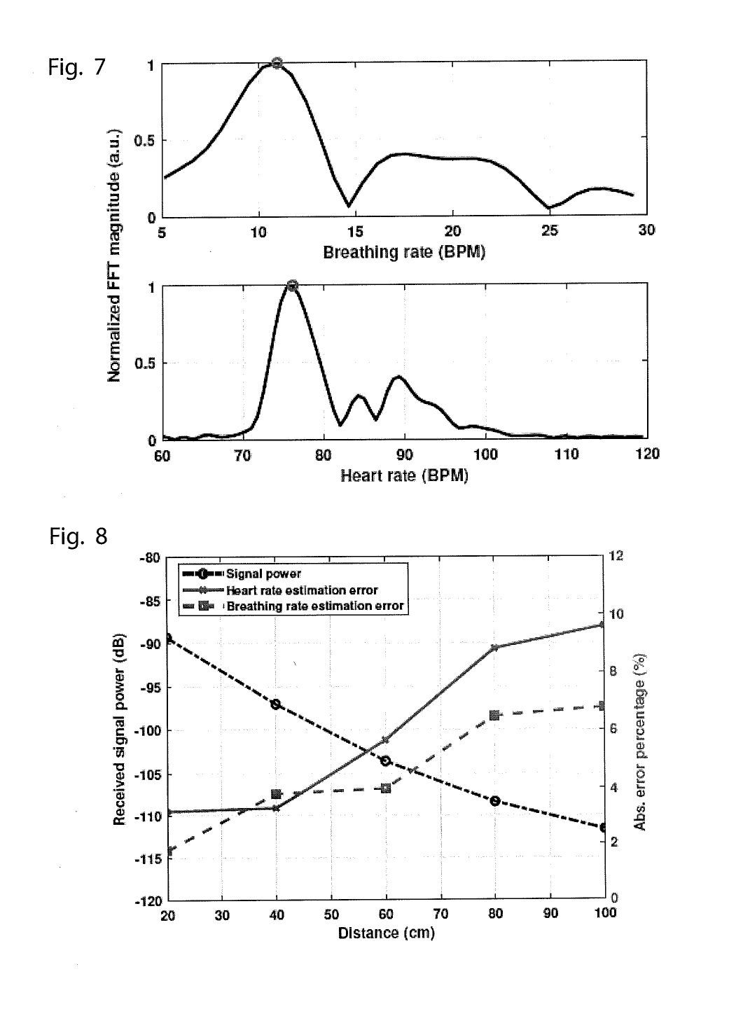

Given the foregoing or similar preprocessing steps, the largest frequency component in the region of interest can be identified (see FIG. 7) (boxes 225 and 245). The previous steps can be carried out multiple times on the same subject to increase the precision and reliability of the measured heart rate and/or respiration rate, subject (in different time windows) and at the end the average of the breathing and/or heart rate during the entire measurement period can be calculated (box 250), although many other approaches to combining the calculated dominant frequencies could be used instead (e.g., median, mode, trimmed mean, etc.).

### Breathing Rate Estimation

Now, turning in more detail to one aspect of an embodiment, a subject's breathing rate can be estimated by determining the highest frequency component in the band of noimal breathing rates for an adult human (this can readily be tailored to other age ranges by those of ordinary skill in the art). First, the raw data (see FIG. 3 for example raw data) received from the PD with an infinite impulse response (IIR) Chebyshev Type 2 band-pass filter (BPF) of appropriate passband for adult breathing rates as seen in FIG. 4. After filtering the data (e.g., FIG. 5), each dataset is multiplied with a Hanning window to remove the spectral leakage before the fast Fourier transform (FFT) process. An FFT works well with infinite data, but to use it with finite data the leakage introduced by avoiding the convolution of the frequency domain data with the rectangular window infinite frequency response should be removed. After the FFT of the data is taken and its frequency spectrum calculated, then the highest power frequency or band in the region of interest is identified. (In certain embodiments the range of 3 breaths/minute to 30 breaths/minute can be used as a search window). In the example of FIG. 4, notice that the difference in frequency content between the heart and breathing rates.

The foregoing steps can be performed on each dataset and at the end, it may be useful to the average breathing rate during the entire measurement time interval can be calculated. Note that if the digital data is not stationary (e.g., if the subject has exercised and then allowed to rest) the length of the data window might be shortened. Finally, the effect of filtering the data is shown in FIG. 7.

### Results and Comparison

Using the setup and algorithms disclosed previously, vitals of a subject have been obtained and compared with the off-the-shelf hardware devices to determine the accuracy of the instant approach. In this trial, the following parameters were utilized:
The duration for each single measurement was 60 seconds and then data were separated into windows for estimation.
The distance between human target and VLS system was 40 cm unless stated otherwise.
The algorithm FFT size (window size) was 2048 (around 20 seconds for ADC sampling rate (Fs)=100 samples per second).

In order to further investiagate the accuracy of an embodiment, multiple subjects were tested and the results compared with the contact-based devices, with the results being posted below in Table 1. In this table, the subject's measured values and their absolute error percentage values with respect to the contact-based devices is compared. of the measurement taken by our system compared to the off-the-shelf contact-based devices for different subjects. It is worth mentioning that the off-the-shelf devices [50] are assumed to be 100% accurate. The results are based on an average value of 10 measurements for each subject. After evaluating our system on four . . . different users. It was observed that an average of 90% accuracy after 40 tests.

| | VLS-based | | Contact-based | Heart rate | VLS-based | | Contact-based | | Breathing rate |
|---|---|---|---|---|---|---|---|---|---|
| Scenario | heart rate | | heart rate | error percentage | breathing rate | | breathing rate | | error percentage |
| Sitting Dark | 86.75 | BPM | 87.1 BPM | 0.40% | 12.5 | BPM | 12.75 | BPM | 1.6% |
| Sitting Normal light | 86.49 | BPM | 87.1 BPM | 0.69% | 11.17 | BPM | 11.55 | BPM | 3.27% |
| Standing Normal light | 83.8 | BPM | 92.6 BPM | 9.47% | 11.34 | BPM | 12.2 | BPM | 6.99% |
| Sleeping Normal light | 79.48 | BPM | 76.5 BPM | 3.90% | 11.19 | BPM | 11.7 | BPM | 4.27% |

Turning next to FIG. 8, this figure examine the effect of altering the distance from the PD to subject has on the accuracy of the estimated heart rate. As expected, as the distance increases the power of the received signal decreases, so the detection of the small fluctuation associated with a heartbeat becomes more difficult and the reliability of the resulting estimate decreases.

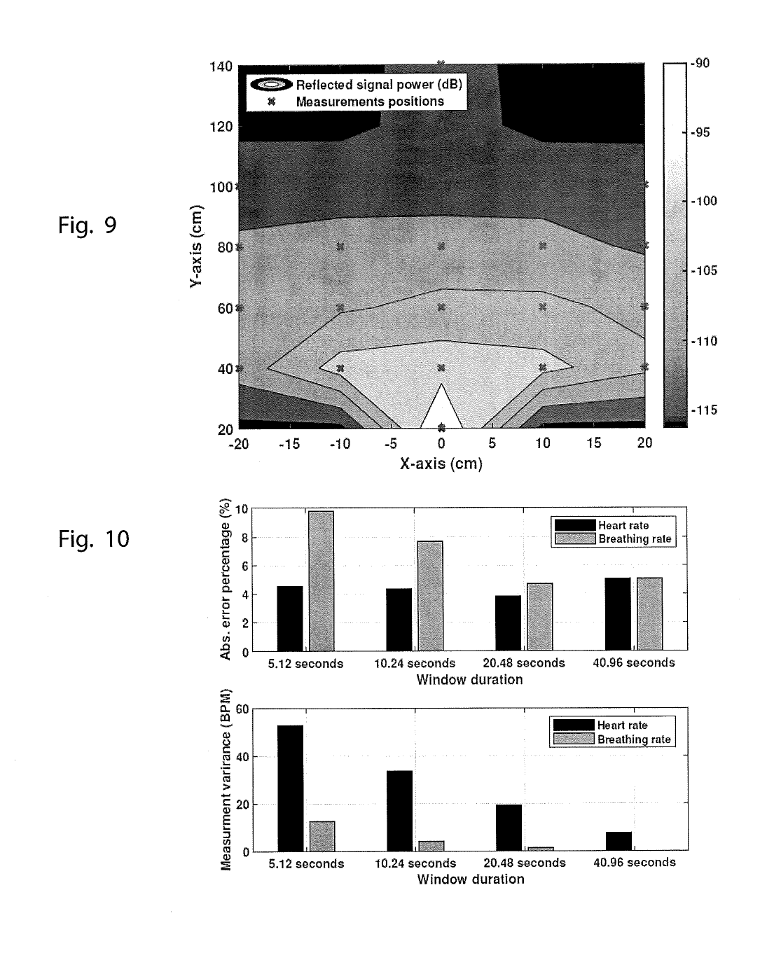

FIG. 9 shows the effect of the changing the position (23 different positions) of the subject and the system where the system is positioned at the origin (0, 0) cm. As the distance and the incidence angle increase the reflected received signal decreases, which will affect the received signal to interference and noise ratio, making the estimation harder and decreasing the accuracy as shown in FIG. 8.

In FIG. 10, the effect of increasing the number of samples used for the estimation accuracy is illustrated. As expected, the estimation error decreases as the data window size increases. This improvement is at least partially attributable to the fact that as the window size is increased the frequency resolution between the different frequency bins at the FFT output decreases. In some cases, if widow size is too long that can lead to an increase in the interference and noise from the other body movements with the result that the estimation error increases and ratio between the heart and breathing frequencies peaks and the noise around them decreases. Moreover, as the data window length increases the estimation variance decreases.

In some embodiments, the best performance in connection with both heart and breathing rate estimation algorithm occurred with a data window of approximately 20 seconds (20.48 seconds) and distance 40 cm.

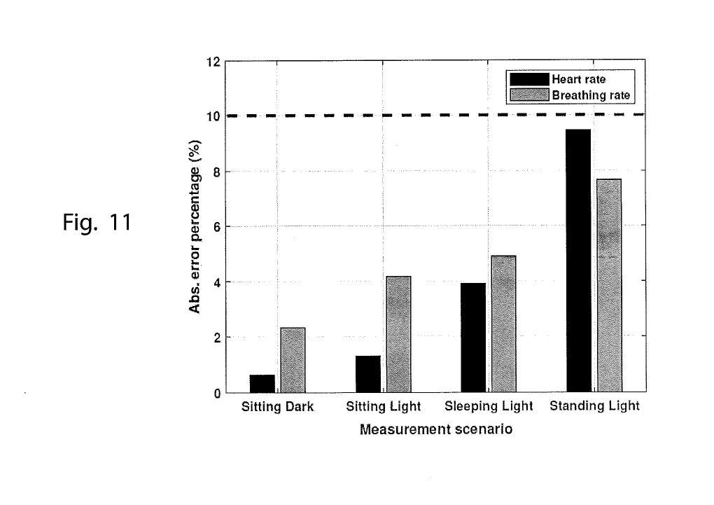

In FIG. 11 the performance of an embodiment of the VLS-based system is demonstrated for three positions of the target subject. In this figure, measurements were taken while the subject was sitting in a chair, sleeping on a table, and standing in front of the instant system. Two lighting conditions were also considered in order to study the effect of lighting on the resulting measurements. It is noted that error percentage in a low-light environment was less than for the comparable measurement under normal lighting conditions. This can be explained by the fact that the noise and interference is much reduced in the dark lighting so it will be easier for the algorithm to detect the heart and breathing rate peaks in the frequency domain signal. In addition to the lighting condition, two scenarios (sleeping and standing) were tested to show how the system can be used in different positions without dramatically changing its accuracy. It is clear that worst scenario is when the human target is standing in front of the system. This finding can be attributed to the increase of uncontrolled target movement during the test which will add additional noise and interference to the received signal and make the estimation worst. Generally, all scenarios have error percentage less than 10% which can be further improved by adding adaptive signal processing and more advanced hardware which will be scenario and setup dependent.

The invention taught herein has potential application in many different patient scenarios where contact-based monitoring might be problematic including, by way of example only, sleep apnea, surgical procedures, sudden-infant-death-syndrome (SIDS), acute heart failure prediction, shock states or respiratory failure, military (defense/protection) and triage applications, disaster medicine, and smart-home health applications, covert monitoring of suspects (e.g., lie detection), anxiety-monitoring for defense and homeland security, and smart-home health applications and real-time vitals monitoring of pilots, drivers (for crash avoidance) and passengers, and human-computer-interaction (HCl) applications.

Although the previous discussion has largely focused on the use of a simple visible light as light source, as noted previously other parts of the light spectrum, e.g., infrared and ultraviolet, could also be used.

According to still another embodiment, the light source(s) could be modulated (via modulation techniques) to attenuate the noise, reduce interference and avoid contamination from of the signal from a DC source, thereby improving the performance and accuracy of that embodiment.

Finally, certain embodiments may use MIMO (multiple-input multiple-output) techniques to improve the performance of those embodiments, i.e., multiple light sources and/or multiple photo-detectors. In some of these embodiments, signals from the multipe photo-detectors might be combined (either digitally or in analog form) to produce a composite signal that is processed as described above. Alternatively, the photo-detectors might be combined in a way that provides a 3D or other view of the imaged part of the body and then subsequently analyzed as discussed above. In stil other variations, the multiple sources might include light sources of different wavelengths or light frequency bands and the resulting analysis could involve summation together or separate analysis of the signals in each wavelength. Those of ordinary skill in the art will devise other ways in which signals from multiple different sources and/or multiple different photo-detectors might be combined to produce an estimate of the target vital sign(s).

By way of comparison with prior art approaches, various embodiments taught herein differ in that these embodiments detect the change of amplitude of the reflected light signal as a noncontact method of measuring patient vital signs.

The peaks of the frequency spectrum of the reflected signal can be found using ambient light (white) although other colors might be used. Still, in various embodiments white light is useful.

It is to be understood that the terms “including”, “comprising”, “consisting” and grammatical variants thereof do not preclude the addition of one or more components, features, steps, or integers or groups thereof and that the terms are to be construed as specifying components, features, steps or integers.

If the specification or claims refer to “an additional” element, that does not preclude there being more than one of the additional element.

It is to be understood that where the claims or specification refer to “a” or “an” element, such reference is not be construed that there is only one of that element.

It is to be understood that where the specification states that a component, feature, structure, or characteristic “may”, “might”, “can” or “could” be included, that particular component, feature, structure, or characteristic is not required to be included.

Where applicable, although state diagrams, flow diagrams or both may be used to describe embodiments, the invention is not limited to those diagrams or to the corresponding descriptions. For example, flow need not move through each illustrated box or state, or in exactly the same order as illustrated and described.

Methods of the present invention may be implemented by performing or completing manually, automatically, or a combination thereof, selected steps or tasks.

The term “method” may refer to manners, means, techniques and procedures for accomplishing a given task including, but not limited to, those manners, means, techniques and procedures either known to, or readily developed from known manners, means, techniques and procedures by practitioners of the art to which the invention belongs.

For purposes of the instant disclosure, the term “at least” followed by a number is used herein to denote the start of a range beginning with that number (which may be a ranger having an upper limit or no upper limit, depending on the variable being defined). For example, “at least 1” means 1 or more than 1. The term “at most” followed by a number is used herein to denote the end of a range ending with that number (which may be a range having 1 or 0 as its lower limit, or a range having no lower limit, depending upon the variable being defined). For example, “at most 4” means 4 or less than 4, and “at most 40%” means 40% or less than 40%. Terms of approximation (e.g., “about”, “substantially”, “approximately”, etc.) should be interpreted according to their ordinary and customary meanings as used in the associated art unless indicated otherwise. Absent a specific definition and absent ordinary and customary usage in the associated art, such terms should be interpreted to be ±10% of the base value.

When, in this document, a range is given as “(a first number) to (a second number)” or “(a first number)-(a second number)”, this means a range whose lower limit is the first number and whose upper limit is the second number. For example, 25 to 100 should be interpreted to mean a range whose lower limit is 25 and whose upper limit is 100. Additionally, it should be noted that where a range is given, every possible subrange or interval within that range is also specifically intended unless the context indicates to the contrary. For example, if the specification indicates a range of 25 to 100 such range is also intended to include subranges such as 26-100, 27-100, etc., 25-99, 25-98, etc., as well as any other possible combination of lower and upper values within the stated range, e.g., 33-47, 60-97, 41-45, 28-96, etc. Note that integer range values have been used in this paragraph for purposes of illustration only and decimal and fractional values (e.g., 46.7-91.3) should also be understood to be intended as possible subrange endpoints unless specifically excluded.

It should be noted that where reference is made herein to a method comprising two or more defined steps, the defined steps can be carried out in any order or simultaneously (except where context excludes that possibility), and the method can also include one or more other steps which are carried out before any of the defined steps, between two of the defined steps, or after all of the defined steps (except where context excludes that possibility).

Further, it should be noted that terms of approximation (e.g., “about”, “substantially”, “approximately”, etc.) are to be interpreted according to their ordinary and customary meanings as used in the associated art unless indicated otherwise herein. Absent a specific definition within this disclosure, and absent ordinary and customary usage in the associated art, such terms should be interpreted to be plus or minus 10% of the base value.

Still further, additional aspects of the instant invention may be found in one or more appendices attached hereto and/or filed herewith, the disclosures of which are incorporated herein by reference as if fully set out at this point.

Thus, the present invention is well adapted to carry out the objects and attain the ends and advantages mentioned above as well as those inherent therein. While the inventive device has been described and illustrated herein by reference to certain preferred embodiments in relation to the drawings attached thereto, various changes and further modifications, apart from those shown or suggested herein, may be made therein by those of ordinary skill in the art, without departing from the spirit of the inventive concept the scope of which is to be determined by the following claims.

What is claimed is:

1. A method of estimating at least one subject vital sign using light reflected from the subject, comprising the steps of:
(a) directing a light source at the subject's body;
(b) positioning a photo-detector to receive the light that is reflected from the subject's body;
(c) using the photo-detector to continuously record an amplitude of said light reflected from the subject's body for a period of time to create a reflected light amplitude time series;
(d) band-pass filtering the reflected light amplitude time series in a predetermined frequency range that depends on the vital sign that is to be estimated; and
(e) analyzing the filtered time series to estimate the subject's at least one vital sign.

2. The method of claim 1, wherein said at least one vital sign that is to be estimated is at least one of a respiration rate and a heart rate.

3. The method of claim 1, wherein said at least one vital sign that is to be estimated is the subject's respiration rate and the reflected light amplitude time series is band-pass filtered to pass frequencies from 3 breaths per minute to 30 breaths per minute.

4. The method of claim 1, wherein said at least one vital sign that is to be estimated is the subject's heart rate and the reflected light amplitude time series is band-pass filtered to pass frequencies from 50 to 160 beats per minute (BPM).

5. The method of claim 1, wherein step (a) comprises the step of:
(1) placing a reflective item on the subject's body;
(a2) directing the light source at the reflective item so that light from the light source is reflected toward the photo-detector.

6. The method of claim 1, wherein the light source is at least one of visible light, infrared light, ultraviolet light.

7. The method of claim 1, wherein the light source is an LED light source.

8. The method of claim 1, wherein step (e) comprises the steps of:
(e1) calculating a Fourier Transform of said band-pass filtered reflected light amplitude time series, and
(e2) determining within said predetermined frequency band a maximum frequency value, thereby estimating the subject's vital sign.

9. A method of estimating at least one subject vital sign, comprising the steps of:
(a) exposing a subject's body to at least one light source;
(b) positioning at least one photo-detector to receive light from the at least one light source that is reflected from the subject;
(c) using the at least one photo-detector to continuously record an amplitude of said light reflected from the subject to create a reflected light amplitude time series;
(d) band-pass filtering the reflected light amplitude time series in a predetermined frequency range, wherein said predetermined frequency range is dependent on the vital sign that is to be estimated; and
(e) analyzing the band-pass filtered time series to estimate the at least one subject's vital sign.

10. The method of claim 9, wherein said at least one vital sign is at least one of a respiration rate and a heart rate.

11. The method of claim 9, wherein said at least one vital sign that is to be estimated is the subject's respiration rate and the reflected light amplitude time series is band-pass filtered to pass frequencies from 3 breaths per minute to 30 breaths per minute.

12. The method of claim 9, wherein said at least one vital sign that is to be measured is the subject's heart rate and the reflected light amplitude time series is band-pass filtered to pass frequencies from 50 to 160 beats per minute (BPM).

13. The method of claim 9, wherein step (a) comprises the step of:
(a1) placing a reflective item on the subject's body;
(a2) positioning the photo-detector so that it can receive light reflected from the reflective item.

14. The method of claim 9, wherein the at least one light source is at least one of visible light, infrared light, ultraviolet light.

15. The method of claim 9, wherein the at least one light source is an LED light source.

16. The method of claim 9, wherein step (e) comprises the steps of:
(e1) calculating a Fourier Transform of said band-pass filtered reflected light amplitude time series, and,
(e2) determining within said predetermined frequency band a maximum frequency value, thereby estimating the subject's vital sign.
